SEQUENCE LISTING (17 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - DNA, 20 bp, Artificial Sequence
  Primer
ggggacatgcagtatgagtt

SEQ ID NO: 3 - DNA, 20 bp, Artificial
  Primer
ggcctggtagttgttgtgag

SEQ ID NO: 4 - DNA, 16 bp, Artificial Sequence
  Primer
gaatcagtcggcccat

SEQ ID NO: 5 - DNA, 18 bp, Artificial Sequence
  Primer
gttcgccagtttattgct

SEQ ID NO: 6 - DNA, 17 bp, Artificial Sequence
  Primer
gagacggcgatagttcc

SEQ ID NO: 7 - DNA, 16 bp, Artificial Sequence
  Primer
agtgccgctaactcaa

SEQ ID NO: 8 - DNA, 19 bp, Artificial Sequence
  Primer
caccccagccgcaaagagt

SEQ ID NO: 9 - DNA, 20 bp, Artificial Sequence
  Primer
cgggcagaaagcacagcact

SEQ ID NO: 10 - DNA, 16 bp, Artificial Sequence
  Primer
gcgcggataaggatga

SEQ ID NO: 11 - DNA, 16 bp, Artificial Sequence
  Primer
gtccccgtggctactc

SEQ ID NO: 12 - DNA, 16 bp, Artificial Sequence
  Primer
cgccaccgagactaaa

SEQ ID NO: 13 - DNA, 16 bp, Artificial Sequence
  Primer
tcatagcgtccgtcat

SEQ ID NO: 14 - DNA, 20 bp, Artificial Sequence
  Primer
accacagtccatgccatcac

SEQ ID NO: 15 - DNA, 20 bp, Artificial Sequence
  Primer
tccaccaccctgttgctgta

SEQ ID NO: 16 - DNA, 16 bp, Artificial Sequence
  Primer
tttacagcctgcaccc

SEQ ID NO: 17 - DNA, 16 bp, Artificial Sequence
  Primer
ctagcagtgacggtct

(19) United States
(12) Patent Application Publication
(10) Pub. No.: US 2020/0323895 A1
(43) Pub. Date: Oct. 15, 2020

(54) Compounds, Compositions, and Methods for Treating And/Or Preventing Periodontal Disease

(71) Applicants: THE UNITED STATES OF AMERICA, AS REPRESENTED BY THE SECRETARY, DEPARTMENT OF HEALTH AND HUMAN SERVIC, Bethesda, MD (US); YALE UNIVERSITY, New Haven, CT (US)

(72) Inventors: Martha Somerman, Bethesda, MD (US); Brian Foster, Bethesda, MD (US); Emily Chu, Bethesda, MD (US); Demetrios Braddock, Guilford, CT (US); Enrique De La Cruz, New Haven, CT (US)

(21) Appl. No.: 16/765,420

(22) PCT Filed: Nov. 27, 2018
(86) PCT No.: PCT/US2018/062593

§ 371 Date: May 19, 2020

Related U.S. Application Data
(60) Provisional application No. 62/590,824, filed on Nov. 27, 2017.

(51) Int. Cl. A61K 31/7076

(52) U.S. Cl. CPC A61K 31/7076; A61K 9/0019

(57) ABSTRACT

A method of treating or preventing periodontal disease in a subject comprises administering to the subject an inhibitor of ecto-nucleotide pyrophosphate/phosphodiesterase-I (ENPP1). An ecto-nucleotide pyrophosphate/phosphodiesterase-I (ENPP1) inhibitor and pharmaceutical compositions for use in treatment or prevention of periodontal disease or for increasing cementum formation are also disclosed.

### CROSS REFERENCE TO RELATED APPLICATIONS

This application claims the benefit of U.S. Provisional Application No. 62/590,824, filed Nov. 27, 2017, incorporated by reference in its entirety herein.

### STATEMENT REGARDING FEDERALLY SPONSORED RESEARCH OR DEVELOPMENT

This disclosure was made with government support under AR041196 and AR041197 awarded by National Institutes of Health. The government has certain rights in the disclosed subject matter.

### BACKGROUND

Periodontal disease, also known as a type of gum disease or periodontitis, comprises a set of inflammatory diseases affecting the periodontium, which are the tissues surrounding and supporting the teeth: gum tissue (or gingiva), cementum (outer layer of the tooth roots), alveolar bone, and periodontal ligaments (connective tissue fibers that run between the cementum and the alveolar bone). Periodontitis is caused by microorganisms that adhere to and grow on the tooth's surfaces, along with an over-aggressive immune response against these microorganisms. Periodontal disease comprises a range of diseases, which may develop sequential or in tandem to include: gingivitis, chronic periodontitis, aggressive periodontitis, periodontitis as a manifestation of systemic disease, necrotizing ulcerative gingivitis/periodontitis, abscesses of the periodontium, and combined periodontic-endodontic lesions. Periodontitis causes progressive loss of the alveolar bone, periodontal ligament around the teeth, as well as destruction of tooth root cementum, and when untreated can lead to loosening and subsequent loss of teeth.

Cementum is an avascular mineralized tissue that anchors the tooth to the periodontal ligament. When cementum is lost, the tooth becomes detached from the periodontal ligament, and the resulting pocket creates a nidus for the chronic infections that characterize periodontal disease. Current treatments of periodontal disease do not address cementum loss at all. Instead, periodontal treatment is centered on oral hygiene, oral medications (e.g. antibiotics, including low dose doxycycline) and localized application of antibiotic to the periodontal “pockets” so as to fight chronic infection in the nidus, which contributes to further degradation of cementum, as well as surgical therapies targeted at reducing pocket depth, regaining root coverage and/or regeneration of alveolar bone. Outcome of regenerative therapies are often not evidenced-based, not robust, and consequently, not predictable.

There is thus a need to identify compounds, compositions, and methods that can be used to treat or prevent periodontal disease or to promote cementogenesis in a robust, evidenced-based, and predictable fashion.

### SUMMARY

Disclosed herein are methods and compositions for treating or preventing periodontal disease.

The method comprises administering to a subject an inhibitor of ecto-nucleotide pyrophosphate/phosphodiesterase-I (ENPP1).

A pharmaceutical composition comprises an ecto-nucleotide pyrophosphate/phosphodiesterase-I (ENPP1) inhibitor for treatment or prevention of periodontal disease or for increasing cementum formation.

Also disclosed is an ecto-nucleotide pyrophosphate/phosphodiesterase-I (ENPP1) inhibitor for use in treatment or prevention of periodontal disease or for increasing cementum formation.

The above described and other features are exemplified by the following figures and detailed description.

### BRIEF DESCRIPTION OF THE DRAWINGS

The following detailed description of illustrative embodiments of the disclosed subject matter will be better understood when read in conjunction with the appended drawings. Although specific embodiments are illustrated in the drawings, it should be understood, however, that the disclosed subject matter is not limited to the precise arrangements and instrumentalities of the embodiments shown in the drawings.

FIG. 1 illustrates certain aspects of tooth and supporting structures, exemplifying certain sites of cementum loss in periodontal disease.

FIG. 2 illustrates a non-limiting exemplary mechanism for regulation of cementum formation via phosphate/pyrophosphate (Pi/PPi) ratio.

FIG. 3A illustrates certain non-limiting in vivo effects of mouse ENPP1-Fc on ENPP1^{asj/asj} mouse cementum. Murine ENPP1 loss of function (LOF) models have increased cementum, and administration of ENPP1-Fc reduces cementum formation, providing in vivo evidence that systemic delivery of ENPP1 is effective at the local tooth root site. In this situation, introducing ENPP1 function into the ENPP1^{asj/asj} mouse decreased cementum formation. In certain embodiments, as seen in FIG. 2, inhibition of ENPP1 activity promotes cementum production (cementogenesis).

FIG. 3B shows micro-computed tomography (CT) quantification of acellular cementum in each of the three populations of mice (n=3-5 in each group). In this graph, 4 asterisks signifies p<0.0001 and 1 asterisk signifies p<0.05.

FIGS. 4A-4B presents representative CT images of a tooth of a GACI patient (FIG. 4B) and a normal human subject (FIG. 4A). The arrows indicate the cememtum layers in each image. Micro-computed tomography (CT) was used to analyze human and mouse teeth. Samples were scanned in a μCT 50 (Scanco Medical) at 70 kVp, 76 μA, 0.5 Al filter, with 900-ms integration and 10-μm voxel dimension for human samples and 1,200-ms integration and 2-μm voxel dimension for mouse samples. Reconstructed images were analyzed using AnalyzePro 1.0 (AnalyzeDirect).

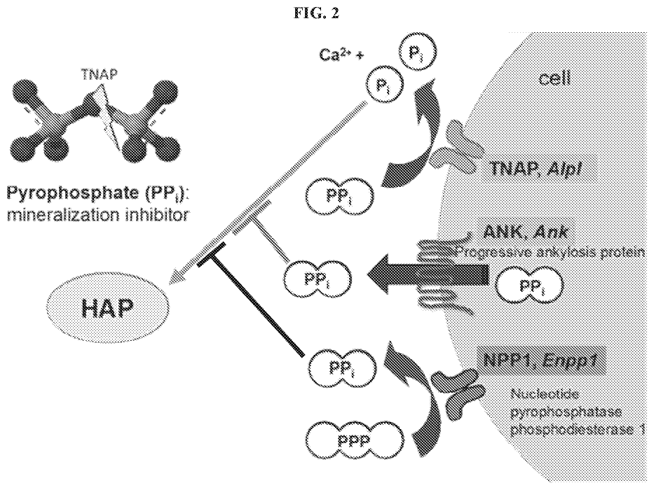

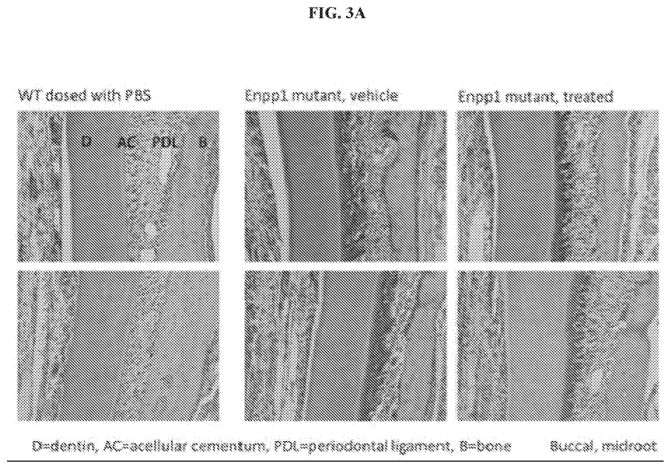

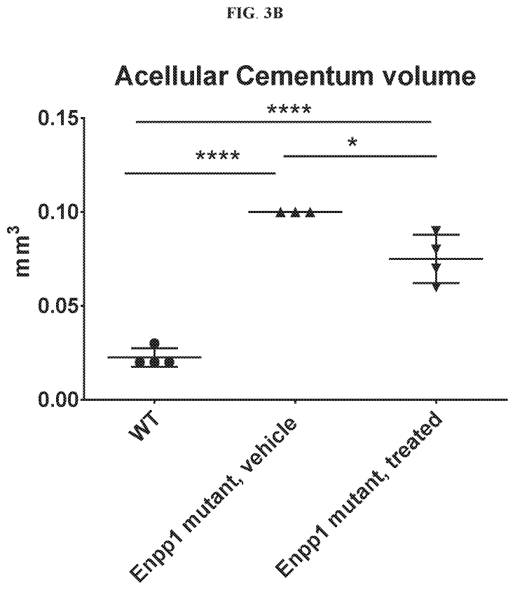

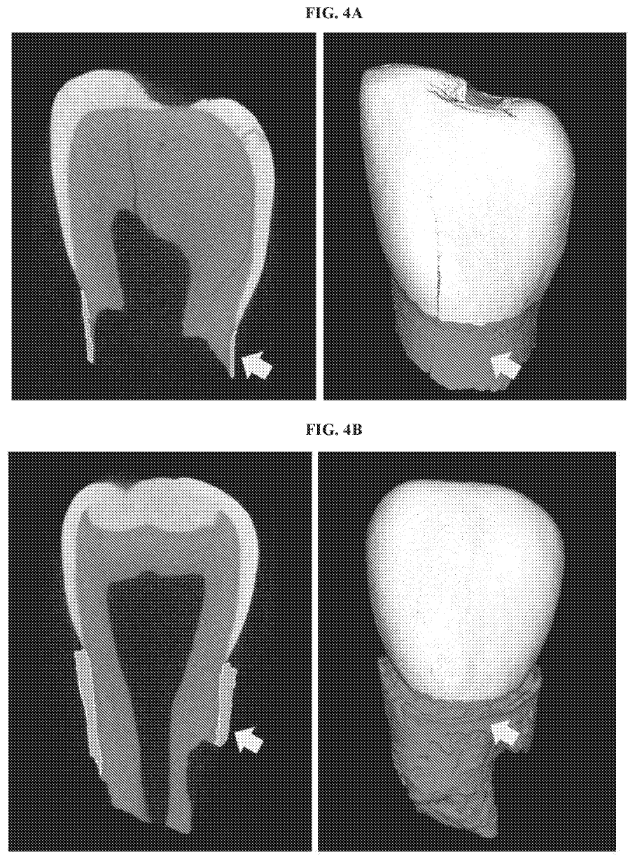

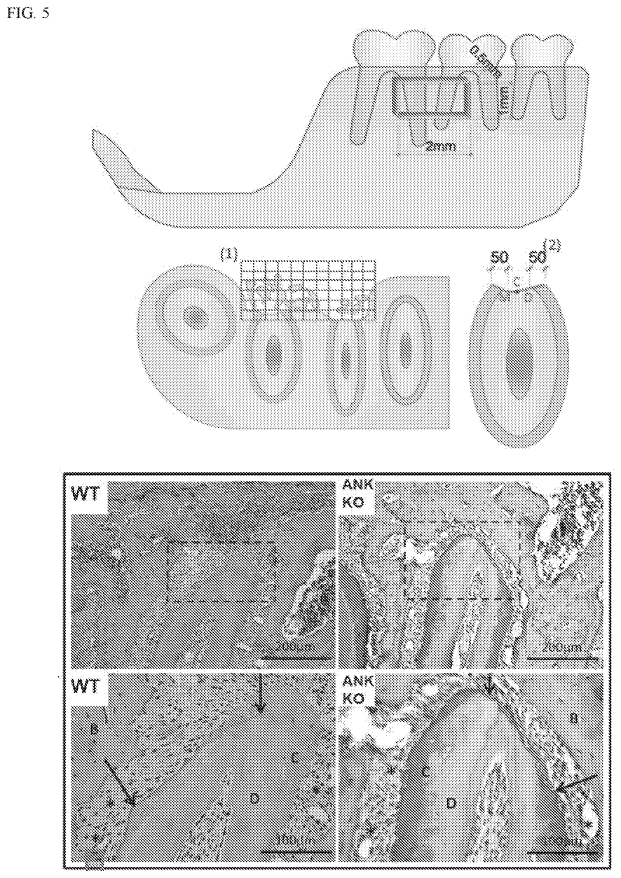

Following data obtained in murine models of ENPP1 loss of function models (FIGS. 3A and 3B), 2-dimensional cross section (left) and 3-dimensional reconstruction (right) microCT images of teeth in GACI patients (FIG. 4B) were compared to normal human controls (FIG. 4A). GACI, an ultra-rare neonatal disease characterized by infantile onset of widespread arterial calcifications in large and medium sized vessels, has also been shown to be characterized by significantly impaired ENPP1 activity and serum plasma pyrophosphate. The cementum layers in GACI patients were much greater, demonstrating that inhibition of ENPP1 in humans results in increased cementum production.

FIG. 5 illustrates a non-limiting animal model of periodontal repair/regeneration (technique schematic (upper) and resultant histology images (lower)), in which efficacy of ENPP1 inhibitors in periodontal disease is tested using the methodology of Rodrigues, et al., 2011, J. Periodontol. 82(12):1757-66.

### DETAILED DESCRIPTION

The inventors have discovered that inhibition of ectonucleotide pyrophosphatase/phosphodiesterase 1 (also known as ENPP1) promotes cementum formation in mammals. Inhibition of ENPP1 can therefore be used to treat periodontal disease in a subject. In certain embodiments, inhibition of ENPP1 can prevent periodontal disease in individuals with genetic defects resulting in lack of or minimal cementum formation, with a goal of increasing cementum formation and subsequently new periodontal ligament (PDL) attachment and alveolar bone formation. The subject can be a mammal. In certain embodiments, the subject is human.

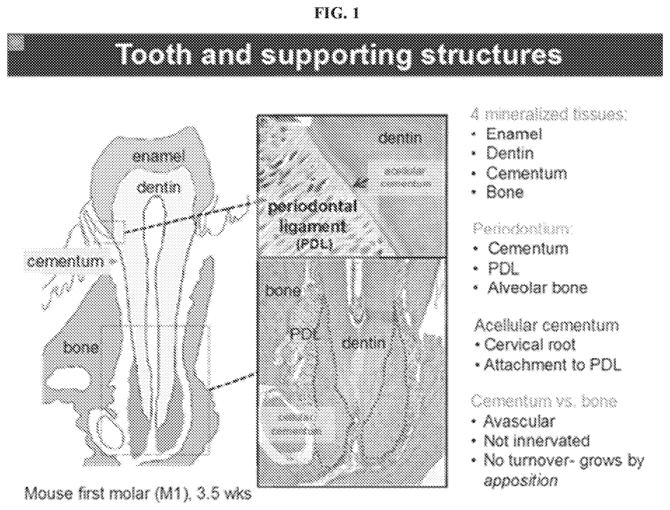

Cementum is an avascular mineralized tooth root structure that demonstrates very limited turnover in vivo and serves to attach the tooth to the periodontal ligament, which has insertions into supporting bone. In humans, and rodents, cementum is located along the root surface, i.e., below the cementum-enamel junction (just under the gums in healthy individuals, FIG. 1). Patients with periodontal disease experience a localized loss of cementum as a result of diseased cementum caused by microbial toxins and host-immune inflammatory responses and results in detachment of the periodontal ligament from the tooth root. There is currently no known method by which cementum production can be promoted such that the loss of cementum in periodontal disease can be replaced. As demonstrated herein, inhibition of ENPP1 activity, using for example small molecule ENPP1 inhibitors, can be used to promote production of cementum, thus treating and/or ameliorating, periodontal disease and even prevent genetically associated periodontal disease related to absence of cementum.

The human ENPP protein family consists of seven extracellular, glycosylated proteins (i.e., ENPP1, ENPP2, ENPP3, ENPP4, ENPP5, ENPP6, and ENPP7) that hydrolyze phosphodiester bonds. ENPPs are cell-surface enzymes, with the exception of ENPP2, which is exported to the plasma membrane but is cleaved by furin and released into the extracellular fluid. The enzymes have high degrees of sequence and structural homology, but exhibit a diverse substrate specificity that encompasses nucleotides to lipids.

ENPP1 is a type II extracellular membrane bound glycoprotein located on the mineral-depositing matrix vesicles of osteoblasts and chondrocytes, as well as the vascular surface of cerebral capillaries and other mineralized tissue associated cells. ENPP1 catabolizes the degradation of extracellular adenosine triphosphate (ATP) into adenosine monophosphate (AMP) and pyrophosphate (PPi), and is the major source of extracellular PPi in the body. PPi inhibits ectopic tissue mineralization, presumably by occupying some of the phosphate (Pi) sites on the surface of nascent or growing hydroxyapatite (HA) crystals, thereby creating irregularities that slow or terminate the propagation of crystal growth and also as a direct effect on osteoclast activity. The construct ENPP1-Fc reduces generalized arterial calcifications in mice homozygous for an ENPP1 mutation (ENPPl^{asi/asi}) (Albright, et al., 2015, Nature Comm. 10006). The ENPPl^{asi/asi} mouse serves as an animal model of generalized arterial calcification of infants (GACI), which is an ultra-rare neonatal disease characterized by infantile onset of widespread arterial calcifications in large and medium sized vessels, resulting in cardiovascular collapse and death in the neonatal period.

As demonstrated herein, cementum is highly sensitive to modulation of Pi/PPi ratio (see, for example, FIGS. 3A-3B). ENPP1 is a major source of extracellular source of PPi, and cementum production is markedly increased in physiologic states of ENPP1 inhibition. In fact, animal models of ENPP1 loss of function (LOF) demonstrated increased cementum production (more than 10 times) at the tooth root, between the tooth and the periodontal ligament (FIG. 3). The animal model of ENPP1 LOF used was the enpp1^{asj/asj} mouse, the accepted animal model of GACI. Treatment of enpp1^{asj/asj} mice with ENPP1-Fc resulted in inhibition of cementum production (FIGS. 3A and 3B). Consistent with these murine results, images of teeth from normal human and human patients with GACI were obtained, confirming that GACI patients with loss of function of ENPP1 have markedly increased cementum (FIGS. 4A-4B). Taken together, these results constitute in vivo evidence that inhibition of ENPP1 increases cementum production in humans. Inhibitors of ENPP1 activity, such as but not limited to small molecule inhibitors of ENPP1, can be used to promote cementum formation in a subject, thus treating periodontal disease in the subject and perhaps preventing periodontal disease in subjects lacking ability to form cementum due to certain rare genetic disorders.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, illustrative methods and materials are described.

As used herein, each of the following terms has the meaning associated with it in this section.

The following notation conventions are applied to the present disclosure for the sake of clarity. In any case, any teaching herein that does not follow this convention is still part of the present disclosure, and can be fully understood in view of the context in which the teaching is disclosed. Protein symbols are disclosed in non-italicized capital letters. As non-limiting examples, ‘ENPP1’ refer to the corresponding protein. In certain embodiments, if the protein is a human protein, an ‘h’ is used before the protein symbol. In other embodiments, if the protein is a mouse protein, an ‘m’ is used before the symbol. Hence, human ENPP1 is referred to as ‘hENPP1’, and mouse ENPP1 is referred to as ‘mENPP1’. Human gene symbols are disclosed in italicized capital letters. As a non-limiting example, the human gene corresponding to the protein hENPP1 is ENPP1. Mouse gene symbols are disclosed with the first letter in upper case and the remaining letters in lower case; further, the mouse gene symbol is italicized. As a non-limiting example, the mouse gene that makes the protein mEnpp1 is Enpp1. Notations about gene mutations are shown as uppercase text. For example, a transgenic mouse with a mutation in the gene Enpp1 that is associated with stiffened joints is called an ‘asj’ mutation and is annotated as Enpp1^{asj/asj} to denote the gene and phenotype associated with the mutation.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes. Mutations encompassed by the present disclosure can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present disclosure may alter splicing the mRNA (splice site mutation) or cause a shift in the reading frame (frameshift).

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, symptom, or cause of a disease, or any other desired alteration of a biological system. An appropriate therapeutic amount in any individual case may be determined by one of ordinary skill in the art using routine experimentation.

As used herein, the term “Fc” refers to a human or mouse IgG Fc domain.

“Instructional material,” as that term is used herein, includes a publication, a recording, a diagram, or any other medium of expression which can be used to communicate the usefulness of the compound of the disclosure in the kit for identifying or alleviating or treating the various diseases or disorders recited herein. Optionally, or alternately, the instructional material may describe one or more methods of identifying or alleviating the diseases or disorders in a cell or a tissue of a subject. The instructional material of the kit may, for example, be affixed to a container that contains the compound of the disclosure or be shipped together with a container that contains the compound. Alternatively, the instructional material may be shipped separately from the container with the intention that the recipient uses the instructional material and the compound cooperatively.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, the term “NPP” or “ENPP” refers to ectonucleotide pyrophosphatase/phosphodiesterase.

As used herein, the term “patient,” “individual” or “subject” refers to a human or a non-human mammal. Non-human mammals include, for example, livestock and pets, such as ovine, bovine, porcine, canine, feline, and murine mammals. In certain embodiments, the patient, individual, or subject is human.

As used herein, the term “prevent” or “prevention” with respect to periodontal disease means no disorder or disease development if none had occurred, or no further disorder or disease development if there had already been development of the disorder or disease. Also considered is the ability of one to prevent some or all of the symptoms associated with the disorder or disease.

As used herein, the term “pharmaceutical composition” refers to a composition comprising at least one compound useful within the disclosed methods, and at least one other substance, such as a carrier, preferably a pharmaceutically acceptable carrier. Pharmaceutical compositions optionally contain one or more additional active agents. The pharmaceutical composition facilitates administration of the compound to a patient. Multiple techniques of administering a compound exist in the art including, but not limited to, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosed subject matter within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosed subject matter, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within disclosed subject matter, and are physiologically acceptable to the patient. Supplementary active compounds may also be incorporated into the compositions. The “pharmaceutically acceptable carrier” may further include a pharmaceutically acceptable salt of the compound useful within disclosed subject matter. Other additional ingredients that may be included in the pharmaceutical compositions used in the practice of the disclosed subject matter are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the disclosed subject matter include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the term “small molecule” refers to a molecule with molecular weight below about 2,000 Da. In certain embodiments, the small molecule is not a polypeptide and/or peptide, but may comprise a polypeptide or protein substructure. In other embodiments, the small molecule is a polypeptide and/or peptide.

As used herein, “substantially purified” refers to being essentially free of other components. For example, a substantially purified polypeptide is a polypeptide that has been separated from other components with which it is normally associated in its naturally occurring state.

As used herein, the term “treatment” or “treating” is defined as the application or administration of a therapeutic agent, i.e., a compound useful within the disclosed subject matter (alone or in combination with another pharmaceutical agent), to a patient, or application or administration of a therapeutic agent to an isolated tissue or cell line from a patient (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

Ranges: throughout this disclosure, various aspects of the disclosed subject matter can be presented in a range format. It should be understood that the description in range format is merely for convenience and brevity and should not be construed as an inflexible limitation on the scope of the disclosed subject matter. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In certain embodiments, the present disclosure contemplates any inhibitor of ENPP1 activity, such as but not limited to any known or yet to be identified small molecule ENPP1 inhibitor, or a solvate or salt (for example, a pharmaceutically acceptable salt) thereof. Inhibitors include non-hydrolyzable analogues of the ENPP1 substrates ATP (adenosine triphosphate) and 2′,3′-cGAMP (Cyclic [G(2′,5′)pA(3′,5′)p]):

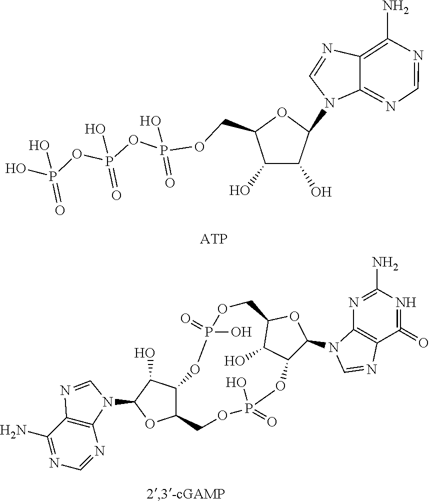

Non-hydrolyzable ATP analogues include α,β-methylene-adenosine 5′-triphosphate (“α,β-methylene-ATP”)

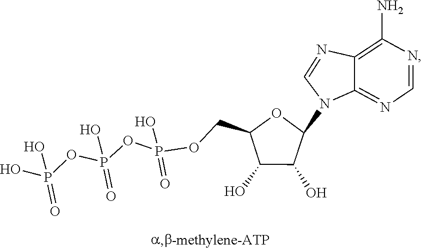 [SMILES: Nc1ncnc2c1ncn2[C@@H]1O[C@H](COP(=O)(O)OP(=O)(O)OP(=O)(O)O)[C@@H](O)[C@H]1O]

(also known as APCP.P, wherein the period denotes a standard O linker between two phosphorous atoms), AP.PNP, AP.PCP, AP.PCCl₂P, APCP.P, ACP.PP, ATPγF, ATPαS, ATPβS, ATPγS, and an analogue, prodrug, salt, or solvate thereof. The structures of these non-hydrolyzable ATP analogues are shown in Table 1. Further examples of non-hydrolyzable ATP analogues are illustrated in Table 2.

TABLE 1
Representation of O-substitution are shown in Table . Further examples
of non-hydrolyzable ATP analogues are illustrated in Table 2.

| | |
|---|---|
| 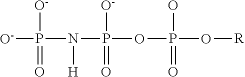 | A [symbol] PNP |
| 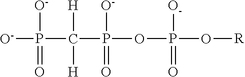 | A [symbol] P [symbol] P |
| 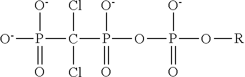 | A [symbol] [symbol] C [symbol] [symbol] |
| 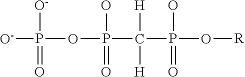 | APC [symbol] P |
| 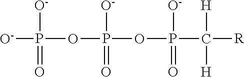 | AC [symbol] PP |
| 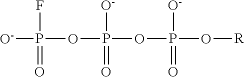 | ATPγF |
| 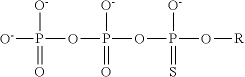 | ATPαS |
| 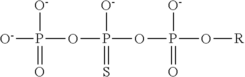 | ATPβS |
| 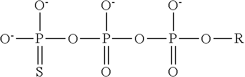 | ATPγS |

[symbol]

indicates data missing or illegible when filed

TABLE 2
ATP analogues

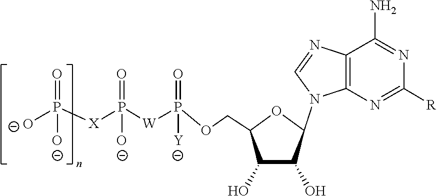

| | | | | | | |
|---|---|---|---|---|---|---|
| 1 | X = CH₂ | Y = O | W = O | R = MeS | n = 1 | |
| 2 | X = CH₂ | Y = BH₃ | W = O | R = H | n = 1 | isomer A |
| 3 | X = CH₂ | Y = BH₃ | W = O | R = H | n = 1 | isomer B |
| 4 | X = CH₂ | Y = BH₃ | W = O | R = MeS | n = 1 | isomer B |
| 5 | X = CH₂ | Y = BH₃ | W = O | R = MeS | n = 1 | isomer A |
| 6 | X = CCl₂ | Y = BH₃ | W = O | R = MeS | n = 1 | isomer A |
| 7 | X = CCl₂ | Y = BH₃ | W = O | R = MeS | n = 1 | isomer B |
| 8 | X = CF₂ | Y = BH₃ | W = O | R = MeS | n = 1 | isomer A |
| 9 | X = CF₂ | Y = BH₃ | W = O | R = MeS | n = 1 | isomer B |
| 10 | X = O | Y = O | W = CF₂ | R = MeS | n = 0 | |
| 11 | X = CF₂ | Y = O | W = O | R = MeS | n = 1 | |
| 12 | X = CCl₂ | Y = BH₃ | W = O | R = H | n = 1 | isomer A |
| 13 | X = CCl₂ | Y = BH₃ | W = O | R = H | n = 1 | isomer B |

Non-hydrolyzable 2′,3′-cGAMP inhibitors include, but are not limited to: 2′,3′-cGAM(PS)₂ (Rp/Sp); 3′-Adenylic acid, P-thioguanylyl-(2′→5′)-, cyclic nucleotide; 2′-Guanylic acid, P-thioadenylyl-(3′→5′)-, cyclic (2′→5′)-nucleotide; 2′-Guanylic acid, adenylyl-(3′→5′)-3′-deoxy-, cyclic 2′→5′-nucleotide; 2′-Guanylic acid, adenylyl-(3′→5′)-, cyclic nucleotide; 3′-Guanylic acid, adenylyl-(3′→5′)-, cyclic nucleotide; or analogue, prodrug, salt, or solvate thereof:

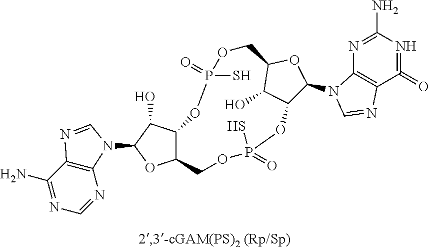

The compounds can be used in treatment or prevention of periodontal disease or to increase cementum formation (cementogenesis).

Compounds that are useful in the disclosed methods can be identified through in vitro and/or in vivo testing, as described elsewhere herein or using any procedure known or recognized in the prior art. In a non-limiting example, compounds useful within the disclosed methods can increase cementum production in cementoblast cultures established in the literature (Rodrigues, et al., 2011, J. Periodontol. 82(12):1757-66; Foster, et al., 2012, PLoS ONE 7(6):e 383-393; Foster, et al., 2011, Cells Tissues Organs 194(5):382-405). In another non-limiting example, compounds useful within the disclosed methods can increase and/or restore cementum production in animal models of periodontal disease established in the literature. These models include murine models of periodontal disease in which periodontal fenestration defects (2 mm/1 mm/0.5 mm) are created on the buccal aspects of mandibular molars in progressive ankylosis protein knock-out (Ank KO) and wild-type (WT) mice, such as described in Rodrigues, et al., 2011, J. Periodontol. 82(12):1757-66 (see FIG. 5). These models also include loss of function murine models of any of the SIBLING (Small Integrin Binding Ligand N-Linked Glycoprotein) family of proteins. For example, loss of function of bone sialoprotein (BSP) in rodent models (BSP knock-out (KO) models) results in a periodontal phenotype, as a consequence of minimal tooth root cementum formation (Foster, et al., 2013, J. Dent. Res. 92(2):166-172). Treatment of BSP KO mice with an ENNP1 inhibitor can be used as a model to determine the ability of the inhibitor to correct the cementum defect.

The compounds useful within the disclosed methods can be formulated as a pharmaceutical composition further comprising at least one pharmaceutically acceptable carrier. In certain embodiments, the pharmaceutical composition is formulated for local administration, i.e., for injection into the diseased site, through the gingival tissue, or delivery after a surgical flap is made, or for buccal administration. In other embodiments, the compound useful within the disclosed methods, is adsorbed or bound to a nanoparticle within the pharmaceutical composition. In yet other embodiments, the compound useful within the disclosed methods, is adsorbed or bound to a nanofiber within the pharmaceutical composition. If surgical procedures are used, then resorbable sutures and/or a wound healing dressing can be used.

### Methods

The disclosure provides methods of treating or preventing periodontal disease, and/or defective cementum formation due to rare genetic deficiencies, in a subject. In certain embodiments, the subject is administered a therapeutically effective amount of at least one compound contemplated within the disclosure. The at least one compound can be formulated as a pharmaceutical composition comprising at least one pharmaceutically acceptable carrier. In yet other embodiments, the at least one compound is applied to and/or injected into the subject's gum tissue. In yet other embodiments, the at least one compound is administered acutely or chronically to the subject. In yet other embodiments, a surgical flap is prepared for delivery of the compound and carrier. In yet other embodiments, the subject is a mammal. In yet other embodiments, the mammal is human.

It will be appreciated by one of skill in the art, when armed with the present disclosure including the methods detailed herein, that the disclosed subject matter is not limited to treatment of a disease or disorder once established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present disclosure may provide benefit. Therefore, the disclosed subject matter, as described more fully herein, includes a method for preventing diseases and disorders in a subject, in that any compound contemplated herein, can be administered to a subject prior to the onset of the disease or disorder, thereby preventing the disease or disorder from developing. One of skill in the art, when armed with the disclosure herein, would appreciate that the prevention of a disease or disorder in a subject encompasses administering to a subject a compound contemplated herein as a preventative measure against a disease or disorder.

The disclosed subject matter encompasses administration of a compound contemplated herein to practice the disclosed methods; the skilled artisan would understand, based on the disclosure provided herein, how to formulate and administer any compound contemplated herein to a subject. However, the disclosed subject matter is not limited to any particular method of administration or treatment regimen. This is especially true where it would be appreciated by one skilled in the art, equipped with the disclosure provided herein, that methods of administering a compound disclosed herein can be determined by one of skill in the pharmacological arts.

### Pharmaceutical Compositions and Formulations

The disclosure provides pharmaceutical compositions comprising at least one compound useful within the disclosed methods. In certain embodiments, the at least one compound is the only therapeutically effective agent present in the composition. In other embodiments, the at least one compound is the only therapeutically effective agent present in the composition in sufficient amount to treat or prevent a disease or disorder contemplated within the disclosed subject matter.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In certain embodiments, the pharmaceutical compositions useful for practicing the disclosed methods may be administered to deliver a dose of between 1 ng/kg/day and 100 mg/kg/day. In other embodiments, the pharmaceutical compositions useful for practicing the disclosed methods may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosed subject matter will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the disclosed methods may be suitably developed for oral, parenteral, topical, transdermal, buccal, ophthalmic, or another route of administration. Other contemplated formulations include projected nanoparticles, liposomal preparations, resealed erythrocytes containing the active ingredient, and immunologically-based formulations. The route(s) of administration is(are) readily apparent to the skilled artisan and depends upon any number of factors including the type and severity of the disease being treated, the type and age of the veterinary or human patient being treated, and the like.

In certain embodiments, the compound and/or composition is/are delivered locally to sites of periodontal disease via chemical and/or ionic attachment or adhesion to suitable substrates currently used for the localized delivery of antibiotic therapy to treat periodontal disease. Examples of substrates used to deliver localized antibiotic therapy to periodontal sites of disease known in the art include, but are not limited to, biofilms, nanoparticles, suture material, microspheres, polymers, fibers, matrixes, and gels. Commercial examples of substrates applied directly to pockets of periodontal disease via methods known in the art and described above include FDA approved products such ARESTIN, ATRIDOX, ACTISITE, PERIOCHIP®, ELYZOL, and DENTOMYSIN.

In certain embodiments, the compound and/or composition is/are administered to the subject so as to minimize or avoid systemic exposure of the subject to the compound and/or composition.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the disclosed pharmaceutical compositions is contemplated include, but are not limited to, humans and other primates, mammals including commercially relevant mammals such as cattle, pigs, horses, sheep, cats, and dogs.

In certain embodiments, the compositions are formulated using one or more pharmaceutically acceptable excipients or carriers. In certain embodiments, the pharmaceutical compositions comprise a therapeutically effective amount of the active agent and a pharmaceutically acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the disclosed pharmaceutical compositions are known in the art and described, for example in Genaro, ed., 1985, Remington's Pharmaceutical Sciences, Mack Publishing Co., Easton, Pa., which is incorporated herein by reference.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives (e.g., sodium carboxymethylcellulose, hydroxypropylmethylcellulose, methylcellulose). Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one that comprises a carbon-containing liquid molecule and which exhibits a less polar character than water. Liquid solutions of the pharmaceutical composition may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

### Administration/Dosing

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the disclosed compositions are administered to the patient in dosages that range from one to five times per day or more. In other embodiments, the compositions are administered to the patient in range of dosages that include, but are not limited to, once every day, every two, days, every three days to once a week, and once every two weeks. It is readily apparent to one skilled in the art that the frequency of administration of the various combination compositions varies from subject to subject depending on many factors including, but not limited to, age, disease or disorder to be treated, gender, overall health, and other factors. Thus, the disclosed subject matter should not be construed to be limited to any particular dosage regime and the precise dosage and composition to be administered to any patient will be determined by the attending physical taking all other factors about the patient into account.

Compounds disclosed for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments therebetween.

In some embodiments, the dose of a compound is from about 0.5 μg and about 5,000 mg. In some embodiments, a dose of a compound used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound disclosed herein, alone or in combination with a second pharmaceutical agent; and instructions for using the compound to treat, prevent, or reduce one or more symptoms of a disease or disorder in a patient.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Routes of Administration

Routes of administration of any of the compositions disclosed herein include localized delivery to periodontal sites of disease, inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the disclosed method are not limited to the particular formulations and compositions that are described herein.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

The present disclosure also includes a multi-layer tablet comprising a layer providing for the delayed release of one or more compounds useful within the methods disclosed herein, and a further layer providing for the immediate release of one or more compounds useful within the methods. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Additional Administration Forms

Additional dosage forms include dosage forms as described in U.S. Pat. Nos. 6,340,475, 6,488,962, 6,451,808, 5,972,389, 5,582,837, and 5,007,790. Additional dosage forms also include dosage forms as described in U.S. Patent Applications Nos. 20030147952, 20030104062, 20030104053, 20030044466, 20030039688, and 20020051820. Additional dosage forms also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions. Thus, single unit dosage forms suitable for oral administration, such as tablets, capsules, gelcaps, and caplets, which are adapted for controlled-release are encompassed by the present disclosure.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” in the context of the disclosure is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the disclosed subject matter may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form. For sustained release, the compounds may be formulated with a suitable polymer or hydrophobic material which provides sustained release properties to the compounds. As such, the compounds for use in the disclosed methods may be administered in the form of microparticles, for example, by injection or in the form of wafers or discs by implantation. In certain embodiments, the compounds are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions or administration regimes, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the disclosed subject matter. However, they are in no way a limitation of the disclosure as set forth herein.

### EXAMPLES

### Methods and Materials:

As previously described (Rodrigues, et al., 2011, J. Periodontol. 82:1757-1766, 2011), and shown in FIG. 5, using mouse models, on one side (left or right mandibular quadrant), a 0.5 cm superficial skin incision (extra oral) is made at the lower border of the mandible, and superficial fascia and muscle is separated to visualize underlying alveolar bone. Periodontal fenestration defects (approximately 2 mm in width, 1 mm in length and 0.5 mm in depth) are created at the buccal aspect of the distal root of first mandibular molars. Superficial bone is removed using a quarter round bur and a high-speed handpiece under saline irrigation. The distal root of the first mandibular molar is denuded of its periodontal ligament, cementum and superficial dentin using a dental chisel and a bur in a slow speed dental handpiece. The ENPP1 inhibitor plus suitable carrier is delivered to the site, and the external incision is sutured. To monitor cementogenesis over time, fluorescent probes are used and injected at designated times, as described in Rodrigues, et al., 2011, J. Periodontol. 82:1757-1766, 2011, and tissues processed at designated times.

The Human ENPP1 Amino Acid Sequence (NCBI accession NP_006199) is SEQ ID NO:1.

### Example 1

### In Vitro Screening:

ENPP1 inhibitors are evaluated in a well-established cementoblast cell line (OCCM cells). Isolation and characterization of OCCM.30 murine cementoblasts has been previously described (D'Errico, et al., 2000, J. Periodontol. 71(1):63-72; Berry, et al., 2003, Connect Tissue Res. 44:Suppl. 1:97-102). Cells are grown in Dulbecco's Modified Eagle Medium (DMEM) with 10% v/v fetal bovine serum (FBS), 2 mM L-glutamine, 100 U/ml penicillin, and 100 μg/ml streptomycin (all reagents from Invitrogen, Carlsbad, Calif.). For gene expression and mineralization experiments, OCCM.30 cells are plated in standard media as described above, with media changed after 24 hrs to DMEM with 1% FBS with 50 μg/ml ascorbic acid (AA). Media are changed every 48 hrs for the remainder of the experiment. Inclusion of organic Pᵢ source β-glycerophosphate (βGP; 5 mM or 10 mM) or 3.3 mM Pi) is used to create mineralizing conditions. ENPP1 inhibitors are added to assay effects on cell function. PGP is purchased from Sigma-Aldrich (St. Louis, Mo.) and a Pi solution is made from sodium phosphate monobasic, monohydrate (NaH2PO4-H2O) and sodium phosphate dibasic, (Na2HPO4-7H20), which are also purchased from Sigma-Aldrich. Cell culture experiments are performed at least three times in triplicates.

Cell proliferation is measured using a non-radioactive, MTS-based assay, following manufacturer's directions (CellTiter 96® AQᵤₑₒᵤₛ proliferation assay, Promega, Madison, Wis.). Absorbance is measured at 570 nm, with reference reading at 750 nm. Absorbance is proportional to the number of living cells in culture.

Production of collagen by cells in vitro is quantified by picrosirius red staining, using methods modified from previous reports (Tullberg-Reinert & Jundt, 1999, Histochem. Cell Biol. 112(4):271-276; Addison & McKee, 2010, Bone 46(4):1100-1107). Briefly, cells are rinsed with PBS and fixed in Bouin's solution for 1 hr at room temperature. The fixative is removed and the plate rinsed several times in water to remove excess Bouin's solution. The collagenous matrix in plates is stained by incubation with picrosirius red dye (Direct Red 80, Polysciences, Inc., Warrington, Pa.) while gently shaking. Unbound dye is removed by rinsing several times with 0.01 N HCl. Bound dye is removed by incubation and shaking with 0.1 N NaOH for at least 1 hr. Picrosirius red is quantified by reading the absorbance at 550 nm. Quantity of collagen is calibrated against a standard curve created by plating and eluting known concentrations of rat tail collagen.

Von Kossa staining for mineral nodule formation is performed using standard procedures (Lillie, 1965, Histopathologic Technique and Practical Histochemistry, McGraw-Hill (Blakiston Division) pp. 172-177). Silver stain was visualized as black, and stain intensity indicated the amount of calcium phosphate precipitation in the cell matrix (silver ions react with phosphate). Cell mineralization in vitro is quantitatively assayed by measuring calcium deposits, using a method modified from a previous report (Addison & McKee, 2010, Bone 46(4):1100-1107). To cell culture wells, 500 μl 0.5 N HCl is added and plates are agitated for 60 min to dissolve calcium-phosphate precipitations. Eluted calcium is measured using a calcium assay (Genzyme Diagnostics, Farmingham, Mass.). One μl of sample is added to 99 μl Arsenazo reagent and absorbance is read at 650 nm. Standard curves are prepared using a calcium stock solution.

A modified assay for measuring in vitro alkaline phosphatase activity (ALP) is used (Osanthanon, et al., 2009, Biomaterials 30(27):4513-21). Briefly, cell cultures are rinsed with PBS and incubated with 200 μl p-Nitrophenyl phosphate (PNPP, Sigma) in the dark at ambient room temperature for 30 min. After incubation, 10 μL supernatant for each condition is transferred to a 96 well plate containing 90 μL of 3 N NaOH (stop solution) per well. Absorbance is recorded at a wavelength of 405 nm.

An enzymatic assay is employed to measure the activity of pyrophosphate-generating ectoenzymes (nucleoside triphosphate pyrophosphohydrolase, NTPPPHase activity), based on a previously described procedure (Rosenthal, et al., 1991, Arthr & Rheumatol. 34(7):904-911). Cells are rinsed with PBS and incubated for 2 hrs with 2.0 ml of 1 mM thymidine 5′-monophosphate p-nitrophenyl ester sodium (TMPNP) solution at 37° C. and without CO₂. After incubation, 20 μL supernatant for each condition is transferred to a 96 well plate containing 80 μL of 0.1 N NaOH (stop solution) per well. Absorbance is recorded at a wavelength of 410 nm.

### RNA Isolation and Real-Time Quantitative RT-PCR

Isolation of RNA, synthesis of cDNA, and performance of real-time quantitative PCR is undertaken as previously described (Foster, et al., 2011, Cells Tissues Organs 194(5):382-405). Total RNA from cells, isolated using the RNeasy Micro kit (Qiagen, Valencia, Calif.) and cDNA, is synthesized from 1.0 μg RNA (Transcriptor kit, Roche Applied Science). PCR reactions are performed with DNA Master SYBR Green I kit (Roche Applied Science) on the Roche Lightcycler 480 system (Roche Diagnostics GmbH, Mannheim, Germany) using intron-spanning primers (www dot gene-expression-analysis dot com). Glyceraldehyde-3-phosphate dehydrogenase (Gapdh) is employed as a housekeeping/reference gene for target gene normalization and relative quantification with amplification efficiency correction. Primer sequences used are listed in Table 3. PCR product identification is performed by post-amplification melting curve analysis. To detect intra- and intergroup gene expression differences, we employ a one-way ANOVA with post-hoc Tukey test, using PASW (SPSS) Statistics 19 software.

TABLE 3
Real time quantitative PCR primer sequences

| | | | For. | | Rev. | |
|---|---|---|---|---|---|---|
| Gene | | | SEQ ID | | SEQ ID | Gene |
| Symbol | Gene Name | Forward (5′-3′) | NO. | Reverse (5′-3′) | NO. | ID |
| Akp2 | Tissue nonspecific | GGGGACATGCAG | 2 | GGCCTGGTAGTTGTTGT | 3 | 11647 |
| | alkaline | TATGAGTT | | GAG | | |
| | phosphatase | | | | | |
| Ank | Progressive | GAATCAGTCGGC | 4 | GTTCGCCAGTTTATTGCT | 5 | 11732 |
| | ankylosis protein | CCAT | | | | |
| Bsp | Bone sialoprotein | GAGACGGCGATA | 6 | AGTGCCGCTAACTCAA | 7 | 15891 |
| | | GTTCC | | | | |
| Col1 | Collagen type 1 | CACCCCAGCCGC | 8 | CGGGCAGAAAGCACAG | 9 | 12842 |
| | alpha 1 | AAAGAGT | | CACT | | |
| Dmp1 | Dentin matrix | GCGCGGATAAGG | 10 | GTCCCCGTGGCTACTC | 11 | 13406 |
| | protein 1 | ATGA | | | | |
| Enpp1 | Ectonucleotide | CGCCACCGAGAC | 12 | TCATAGCGTCCGTCAT | 13 | 18605 |
| | pyrophosphatase | TAAA | | | | |
| | phosphodisetrase 1 | | | | | |
| Gapdh | Glyceraldehyde-3 | ACCACAGTCCAT | 14 | TCCACCACCCTGTTGCT | 15 | 14433 |
| | phosphate | GCCATCAC | | GTA | | |
| | dehydrogenase | | | | | |
| Opn | Osteopontin | TTTACAGCCTGCA | 16 | CTAGCAGTGACGGTCT | 17 | 20750 |
| | | CCC | | | | |

### Example 2. In Vivo Effects of mENPP1-Fc on ENPP1^{asj/asj} Mouse Cementum

Enpp1^{asj/asj} mice, a murine ENPP1 loss of function (LOF) model, are homozygous for a missense mutation in the Enpp1 gene, producing a mutation of V246D in the ENPP1 protein that results in lack of ENPP1 enzymatic activity and therefore in reduced pyrophosphate levels compared to mice of the same genetic background with wild-type (WT) ENPP1.

Mouse ENPP1-Fc (mENPP1-Fc) is a fusion protein of the extracellular domain of mouse ENPP1 and the Fc region of mouse IgG1. Mouse ENPP1-Fc was formulated in vehicle such that the volume of vehicle delivered was 16 ml vehicle per gram of body weight. Vehicle consisted of 10×PBS concentrate (AmericanBio, Inc. Stock # AB11072) diluted to 1× with endotoxin free water and supplemented with 14 mM CaCl₂ and 14 mM ZnCl₂.

Wild-type (WT) ENPP1 or Enpp1^{asj/asj} mice were dosed either with vehicle or with mENPP1-Fc formulated in vehicle. Mice were dosed with daily subcutaneous injections starting at day 14 of life at dose levels of 500 a.u. kg⁻¹ mENPP1-Fc.

Animals were dosed according to total activity of enzyme delivered rather than concentration of enzyme to account for varying activity among batches of enzyme used. One activity unit (1 a.u.) is defined as pM of pNP-TMP substrate hydrolyzed min⁻¹ mg⁻¹ enzyme. The activity assay was performed in a buffer consisting of 50 mM Tris pH9, 150 mM NaCl, 0.1 mM ZnCl₂, 0.1 mM CaCl₂, 0.1 mM MgCl₂. The activity of acceptable protein preparations varied between 40 and 43 a.u. mg⁻¹, and preparations with <40 a.u. mg⁻¹ were discarded. A dose of 500 a.u. kg⁻¹ corresponds to between 6 and 10 mg kg⁻¹, depending on the specific activity of the protein preparation.

FIG. 3A shows representative images of response to administration of mouse ENPP1-Fc to Enpp1^{asj} mice in general accordance with procedures described in Albright, et al., 2015, Nature Comm. 10006. Animals were sacrificed at 35-37 days postnatal. Mandibles were dissected, fixed, and processed for histology or microCT analysis. Standard hematoxylin and eosin staining was used to visualize the periodontal complex. Mandibular first molars were scanned with 2 micron voxel resolution in order to differentiate acellular cementum from dentin. Acellular cementum volume was quantified (n=3-5 for each group), and one way ANOVA (GraphPad Prism) was used to test for statistical significance. The first column of FIG. 3A shows histological images from mice with wild-type (WT) ENPP1 before (upper) and after (lower) administration of vehicle. The second and third columns of FIG. 3 are images from ENPP1^{asj/asj} mice (“ENPP”) before (upper) and after (lower) administration of vehicle (second column) or mENPP1-Fc (third column). The Enpp1^{asj/asj} mice before treatment with mENPP1-Fc have increased cementum (AC) compared to mice with wild-type ENPP1 (WT”). FIG. 3B shows microCT quantification of acellular cementum verifying that ENPP1-Fc administration reduced cementum formation in Enpp1^{asj/asj} mice. Administration of mENPP1-Fc to the Enpp1^{asj} mice introduces ENPP1 enzymatic activity and reduces cementum formation, providing in vivo evidence that systemic delivery of ENPP1 and the resultant increase in PPi level is effective in reducing cementum formation at the local tooth root site.

The disclosure is further illustrated by the following aspects, which are non-limiting.

Aspect 1A: A method of treating or preventing periodontal disease in a subject, the method comprising administering to the subject an inhibitor of ecto-nucleotide pyrophosphate/phosphodiesterase-I (ENPP1).

Aspect 1B: A pharmaceutical composition comprising an ecto-nucleotide pyrophosphate/phosphodiesterase-I (ENPP1) inhibitor, formulated for treatment or prevention of periodontal disease or for increasing cementum formation.

Aspect 1C: An ecto-nucleotide pyrophosphate/phosphodiesterase-I (ENPP1) inhibitor for use in treatment or prevention of periodontal disease or for increasing cementum formation.

Aspect 2: The method, composition, or ENPP1 inhibitor of any one of aspects 1A-1C, wherein the ENPP1 inhibitor comprises a small molecule inhibitor, or an analogue, derivative, prodrug, solvate, or salt thereof.

Aspect 3: The method, composition, or ENPP1 inhibitor of any one of the preceding aspects, wherein the ENPP1 inhibitor comprises a non-hydrolyzable analogue or derivative of ATP or 2′,3′-cGAMP, or a prodrug, solvate, or salt thereof.

Aspect 4: The method, composition, or ENPP1 inhibitor of aspect 3, wherein the non-hydrolyzable analogue or derivative of ATP is at least one compound selected from Table 1 or Table 2.

Aspect 5: The method, composition, or ENPP1 inhibitor of aspect 3, wherein the non-hydrolyzable analogue or derivative of 2′,3′-cGAMP is 2′,3′-cGAM(PS)₂ (Rp/Sp); 3′-Adenylic acid, P-thioguanylyl-(2′→5′)-, cyclic nucleotide; 2′-Guanylic acid, P-thioadenylyl-(3′→5′)-, cyclic (2′→5′)-nucleotide; 2′-Guanylic acid, adenylyl-(3′→5′)-3′-deoxy-, cyclic 2′→5′-nucleotide; 2′-Guanylic acid, adenylyl-(3′→5′)-, cyclic nucleotide; 3′-Guanylic acid, adenylyl-(3′→5′)-, cyclic nucleotide; or a combination thereof.

Aspect 6: The method, composition, or ENPP1 inhibitor of any one of the preceding aspects, wherein the ENPP1 inhibitor is adsorbed or bound to a nanoparticle, nanofiber, suture material, microsphere, polymer, fiber, matrix, gel, or a combination thereof

Aspect 7: The method, composition, or ENPP1 inhibitor of any one of the preceding aspects, wherein the ENPP1 inhibitor is formulated as a pharmaceutical composition.

Aspect 8: The method, composition, or ENPP1 inhibitor of any one of the preceding aspects, wherein the pharmaceutical composition further comprises at least one pharmaceutically acceptable carrier.

Aspect 9: The method, composition, or ENPP1 inhibitor of any one of the preceding aspects, wherein the ENPP1 inhibitor is formulated for injection in gum tissue, local delivery at a surgical flap, buccal delivery, delivery by a resorbable suture, delivery by a wound healing dressing, or a combination of the foregoing.

Aspect 10: The method, composition, or ENPP1 inhibitor of any one of the preceding aspects, wherein the ENPP1 inhibitor is administered acutely or chronically to the subject.

Aspect 11: The method, composition, or ENPP1 inhibitor of any one of the preceding aspects, wherein the subject is a mammal.

Aspect 12: The method, composition, or ENPP1 inhibitor of aspect 11, wherein the mammal is human.

Aspect 13: The method, composition, or ENPP1 inhibitor of any one of the preceding aspects, wherein treating or preventing periodontal disease comprises increasing cementum formation in the subject.

Aspect 14: The method, composition, or ENPP1 inhibitor of any one of the preceding aspects, comprising preventing periodontal disease in a subject with a genetic condition resulting in lack of or minimal cementum formation.

Aspect 15: The method, composition, or ENPP1 inhibitor of any one of the preceding aspects, comprising treating periodontal disease in a subject.

Aspect 16: The method, composition, or ENPP1 inhibitor of any one of the preceding aspects, wherein the ENPP1 inhibitor is present or administered in a therapeutically effective amount.

In general, the compositions and methods described here can alternatively comprise, consist of, or consist essentially of, any components or steps herein disclosed. The compositions and methods can additionally, or alternatively, be manufactured or conducted so as to be devoid, or substantially free, of any ingredients, steps, or components not necessary to the achievement of the function or objectives of the present claims.

Unless defined otherwise, technical and scientific terms used herein have the same meaning as is commonly understood by one of skill in the art to which this disclosure belongs.

The disclosures of each and every patent, patent application, and publication cited herein are hereby incorporated herein by reference in their entirety. However, if a term in the present application contradicts or conflicts with a term in the incorporated reference, the term from the present application takes precedence over the conflicting term from the incorporated reference.

While the disclosed subject matter has been described with reference to specific embodiments, it is apparent that other embodiments and variations may be devised by others skilled in the art without departing from the true spirit and scope of the disclosed subject matter. Additional features known in the art likewise can be incorporated. Moreover, although individual features of some embodiments of the disclosed subject matter can be discussed herein and not in other embodiments, it should be apparent that individual features of some embodiments can be combined with one or more features of another embodiment or features from a plurality of embodiments. The appended claims are intended to be construed to include all such embodiments and equivalent variations.

1. A method of treating or preventing periodontal disease in a subject, the method comprising administering to the subject a therapeutically effective amount of an inhibitor of ecto-nucleotide pyrophosphate/phosphodiesterase-I (ENPP1).

2. The method of claim 1, wherein the ENPP1 inhibitor comprises a small molecule inhibitor, or prodrug, solvate, or salt thereof.

3. The method of claim 1, wherein the ENPP1 inhibitor comprises a non-hydrolyzable analogue of ATP or 2′,3′-cGAMP, or a prodrug, solvate, or salt thereof.

4. The method of claim 3, wherein the non-hydrolyzable analogue of ATP is at least one compound selected from 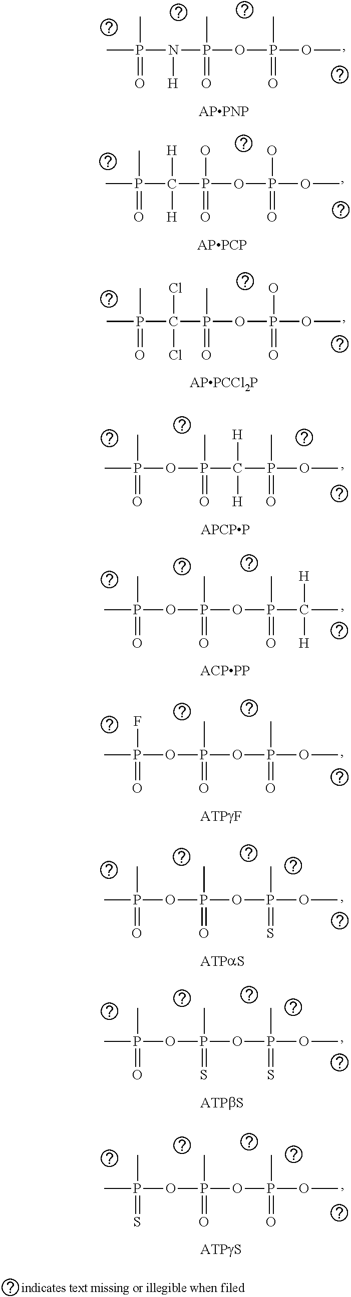
wherein R=5′-adenosinyl group; and
ATP analogues

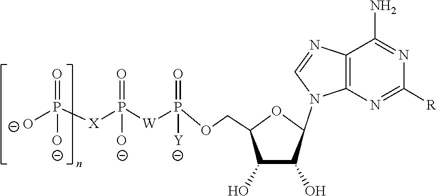

| | | | | | | |
|---|---|---|---|---|---|---|
| 1 | X = CH₂ | Y = O | W = O | R = MeS | n = 1 | |
| 2 | X = CH₂ | Y = BH₃ | W = O | R = H | n = 1 | isomer A |
| 3 | X = CH₂ | Y = BH₃ | W = O | R = H | n = 1 | isomer B |
| 4 | X = CH₂ | Y = BH₃ | W = O | R = MeS | n = 1 | isomer B |
| 5 | X = CH₂ | Y = BH₃ | W = O | R = MeS | n = 1 | isomer A |
| 6 | X = CCl₂ | Y = BH₃ | W = O | R = MeS | n = 1 | isomer A |
| 7 | X = CCl₂ | Y = BH₃ | W = O | R = MeS | n = 1 | isomer B |
| 8 | X = CF₂ | Y = BH₃ | W = O | R = MeS | n = 1 | isomer A |
| 9 | X = CF₂ | Y = BH₃ | W = O | R = MeS | n = 1 | isomer B |
| 10 | X = O | Y = O | W = CF₂ | R = MeS | n = 0 | |
| 11 | X = CF₂ | Y = O | W = O | R = MeS | n = 1 | |
| 12 | X = CCl₂ | Y = BH₃ | W = O | R = H | n = 1 | isomer A |
| 13 | X = CCl₂ | Y = BH₃ | W = O | R = H | n = 1 | isomer B |

5. The method of claim 3, wherein the non-hydrolyzable analogue of 2′,3′-cGAMP is 2′,3′-cGAM(PS)₂ (Rp/Sp); 3′-Adenylic acid, P-thioguanylyl-(2′→5′)-, cyclic nucleotide; 2′-Guanylic acid, P-thioadenylyl-(3′→5′)-, cyclic (2′→5′)-nucleotide; 2′-Guanylic acid, adenylyl-(3′→5′)-3′-deoxy-, cyclic 2′-5′-nucleotide; 2′-Guanylic acid, adenylyl-(3′→5′)-, cyclic nucleotide; 3′-Guanylic acid, adenylyl-(3′→5′)-, cyclic nucleotide; or a combination thereof.

6. The method of claim 1, wherein the ENPP1 inhibitor is adsorbed or bound to a nanoparticle, nanofiber, suture material, microsphere, polymer, fiber, matrix, gel, or a combination thereof.

7. The method of claim 1, wherein the ENPP1 inhibitor is formulated as a pharmaceutical composition.

8. The method of claim 7, wherein the pharmaceutical composition further comprises at least one pharmaceutically acceptable carrier.

9. The method of claim 1 wherein the ENPP1 inhibitor is formulated for injection in gum tissue, local delivery at a surgical flap, buccal delivery, delivery by a resorbable suture, delivery by a wound healing dressing, or a combination of the foregoing.

10. The method of claim 1, wherein the ENPP1 inhibitor is administered acutely or chronically to the subject.

11. The method of claim 1 wherein the subject is a mammal.

12. The method of claim 11, wherein the mammal is a human.

13. The method of claim 1, wherein treating or preventing periodontal disease comprises increasing cementum formation in the subject.

14. The method of claim 1 comprising preventing periodontal disease in a subject with a genetic condition resulting in lack of or minimal cementum formation.

15. A pharmaceutical composition comprising an ecto-nucleotide pyrophosphate/phosphodiesterase-I (ENPP1) inhibitor in an amount effective for treatment or prevention of periodontal disease or for increasing cementum formation.

16. The pharmaceutical composition of claim 15, wherein the composition is formulated for injection in gum tissue, local delivery at a surgical flap, buccal delivery, delivery by a resorbable suture, delivery by a wound healing dressing, or a combination of the foregoing.

17.-21. (canceled)

22. A method of increasing cementum in a subject, the method comprising administering to the subject a therapeutically effective amount of an inhibitor of ecto-nucleotide pyrophosphate/phosphodiesterase-I (ENPP1).

23. The method of claim 22, wherein the ENPP1 inhibitor comprises a small molecule inhibitor, or an analogue, derivative, prodrug, solvate, or salt thereof.

24. The method of claim 22, wherein the ENPP1 inhibitor comprises a non-hydrolyzable analogue of ATP or 2′,3′-cGAMP, or a prodrug, solvate, or salt thereof.

25. The pharmaceutical composition of claim 24, wherein the non-hydrolyzable analogue of ATP is at least one compound selected from 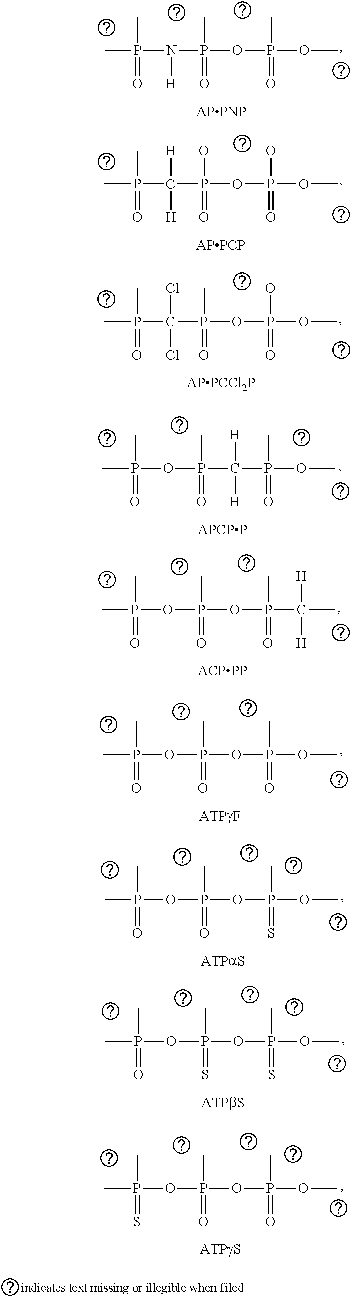
wherein R=5′-adenosinyl group; and
ATP analogues

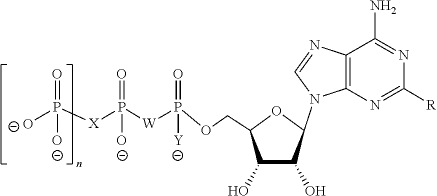

| | | | | | | |
|---|---|---|---|---|---|---|
| 1 | X = CH₂ | Y = O | W = O | R = MeS | n = 1 | |
| 2 | X = CH₂ | Y = BH₃ | W = O | R = H | n = 1 | isomer A |
| 3 | X = CH₂ | Y = BH₃ | W = O | R = H | n = 1 | isomer B |
| 4 | X = CH₂ | Y = BH₃ | W = O | R = MeS | n = 1 | isomer B |
| 5 | X = CH₂ | Y = BH₃ | W = O | R = MeS | n = 1 | isomer A |
| 6 | X = CCl₂ | Y = BH₃ | W = O | R = MeS | n = 1 | isomer A |
| 7 | X = CCl₂ | Y = BH₃ | W = O | R = MeS | n = 1 | isomer B |
| 8 | X = CF₂ | Y = BH₃ | W = O | R = MeS | n = 1 | isomer A |
| 9 | X = CF₂ | Y = BH₃ | W = O | R = MeS | n = 1 | isomer B |
| 10 | X = O | Y = O | W = CF₂ | R = MeS | n = 0 | |
| 11 | X = CF₂ | Y = O | W = O | R = MeS | n = 1 | |
| 12 | X = CCl₂ | Y = BH₃ | W = O | R = H | n = 1 | isomer A |
| 13 | X = CCl₂ | Y = BH₃ | W = O | R = H | n = 1 | isomer B |

and
wherein the non-hydrolyzable analogue or derivative of 2′,3′-cGAMP is 2′,3′-cGAM(PS)2 (Rp/Sp); 3′-Adenylic acid, P-thioguanylyl-(2′→5′)-, cyclic nucleotide; 2′-Guanylic acid, P-thioadenylyl-(3′→5′)-, cyclic (2′→5′)-nucleotide; 2′-Guanylic acid, adenylyl-(3′→5′)-3′-deoxy-, cyclic 2′→5′-nucleotide; 2′-Guanylic acid, adenylyl-(3′→5′)-, cyclic nucleotide; 3′-Guanylic acid, adenylyl-(3′→5′)-, cyclic nucleotide; or a combination thereof.
